(19) United States
(12) Patent Application Publication
(10) Pub. No.: US 2021/0040151 A1
(43) Pub. Date: Feb. 11, 2021

(54) MICROMOLECULE POLYPEPTIDE AND USE THEREOF

(71) Applicant: NANFANG HOSPITAL OF SOUTHERN MEDICAL UNIVERSITY, Guangdong Province (CN)

(72) Inventors: Lili ZHOU, Guangdong Province (CN); Qian YUAN, Guangdong Province (CN)

(73) Assignee: NANFANG HOSPITAL OF SOUTHERN MEDICAL UNIVERSITY, Guangdong Province (CN)

(21) Appl. No.: 16/779,951

(22) Filed: Feb. 3, 2020

(30) Foreign Application Priority Data
Aug. 7, 2019 (CN) 2019107260341

(51) Int. Cl. C07K 7/06; A61K 38/00

(52) U.S. Cl. CPC C07K 7/06; A61K 38/00

(57) ABSTRACT

The invention discloses a micromolecule polypeptide and use thereof. The micromolecule polypeptide KP-6T has a function of significantly inhibiting and reversing kidney tissue fibrosis and CKD progress without obvious toxic and side effects, so that it can be used for manufacturing a medicament for effectively inhibiting the CKD progress. Compared with a micromolecule polypeptide KP-6 disclosed in the prior art, both the KP-6T and the KP-6 can delay kidney tissue fibrosis progress of an advanced UUO mouse, however, the KP-6T contains only 11 amino acids, and has the advantage of shorter peptide chain, easier synthesis, lower cost and easier absorption and distribution in vivo.

### FIELD OF THE INVENTION

The present invention relates to the field of biological medicine, and more particularly, to a micromolecule polypeptide and use thereof in the manufacture of a medicament for treating chronic kidney disease (CKD).

### BACKGROUND OF THE INVENTION

Chronic kidney disease (CKD) is a chronic disease characterized by a change in a kidney structure and a decline in a kidney function caused by various disease causes such as hypertension, diabetes and nephritis for a long period of time (more than three months). A CKD patient whose disease progresses to end-stage renal disease (ESRD) relies on “kidney replacement therapy” for sustaining life. Studies have shown that aging is an independent risk factor for occurrence and development of the CKD. In the past few decades, with the aging of the population in human society, a prevalence rate of the CKD has been on the rise year by year (Nat Rev Nephrol, 2011, 7: 684-696). The CKD is becoming a global “public health problem”, seriously endangering human health and consuming a large number of health resources. However, at present, there are no medicaments for effectively delaying the CKD progress. In view of a pathogenesis of the CKD, finding the medicaments for effectively inhibiting or delaying the CKD progress is undoubtedly a top priority in current kidney disease field, and becomes one of strategic priorities to be tackled urgently.

Kidney fibrosis is a common pathological manifestation of the CKD progressing to a final stage, and is characterized by glomerular sclerosis, kidney tubular atrophy, infiltration of a large number of inflammatory cells in renal interstitium and excessive deposition of extracellular matrix, and thinning of blood capillary. A large number of studies have shown that continuous activation of a Wnt/β-catenin signal after kidney injury is a critical path for the occurrence and development of the CKD and the kidney fibrosis (Kidney Int Supple 2014, 4: 84-90). Moreover, many literatures have disclosed that the activation of the Wnt/β-catenin signal is closely related to liver, lung and heart fibrosis. Therefore, finding a countermeasure to block a Wnt/β-catenin signal pathway has an important theoretical significance and a clinical application value for inhibiting the occurrence and development of kidney, liver, lung and heart fibrosis.

CN106822865A discloses use of a micromolecule polypeptide KP-6, and more particularly, relates to use of the micromolecule polypeptide KP-6 in the manufacture of a medicament for treating chronic kidney disease (CKD). The micromolecule polypeptide KP-6 has an effect of significantly inhibiting kidney tissue fibrosis and CKD progress without obvious toxic and side effects, so that the KP-6T can be used for manufacturing a medicament for effectively treating chronic kidney disease.

### SUMMARY OF THE INVENTION

The present invention is intended to provide a micromolecule polypeptide KP-6T and use thereof in the manufacture of a medicament for treating chronic kidney disease (CKD).

The technical solutions adopted in the present invention are as follows.

The present invention further shortens a peptide chain length based on a micromolecule polypeptide KP-6 disclosed in CN106822865A to obtain the micromolecule polypeptide KP-6T with a length of only 11 amino acids.

A micromolecule polypeptide, comprising any one of the following amino acid sequence:
a) an amino acid sequence shown in SEQ ID NO:1, wherein SEQ ID NO:1 is LQDAYGGWANR; and
b) an amino acid sequence obtained by modifying, substituting, deleting or adding at least one amino acid of the amino acid sequence shown in SEQ ID NO:1.

Use of the micromolecule polypeptide above-mentioned in the manufacture of a reagent for inhibiting expression levels of a β-catenin protein and a downstream target gene thereof.

Further, the downstream target gene of the β-catenin protein includes but is not limited to an I-type plasminogen activator inhibiting factor (PAI-1), Snail-1 and MMP7.

Use of the micromolecule polypeptide above-mentioned in the manufacture of a medicament for treating organ fibrosis, wherein the organ is any one or more selected from the group consisting of kidney, liver, lung and heart.

Use of the micromolecule polypeptide above-mentioned in the manufacture of a medicament for treating chronic kidney disease.

A reagent for inhibiting expression levels of a β-catenin protein and a downstream target gene thereof, comprising an effective dose of the micromolecule polypeptide above-mentioned.

Further, the reagent further comprises a pharmaceutically acceptable ingredient.

A medicament for treating organ fibrosis, wherein the organ is any one or more selected from the group consisting of kidney, liver, lung and heart, and the medicament comprises an effective dose of the micromolecule polypeptide above-mentioned.

Further, the medicament further comprises a pharmaceutically acceptable ingredient.

A medicament for treating chronic kidney disease, comprising an effective dose of the micromolecule polypeptide above-mentioned.

Further, the medicament further comprises a pharmaceutically acceptable ingredient.

The present invention has the beneficial effects as follow.

The present invention provides a micromolecule polypeptide KP-6T. The KP-6T has a function of significantly inhibiting and reversing kidney tissue fibrosis and CKD progress without obvious toxic and side effects, so that it can be used for manufacturing a medicament for effectively inhibiting the CKD progress.

Compared with a micromolecule polypeptide KP-6 disclosed in the prior art, both the KP-6T and the KP-6 can delay kidney tissue fibrosis progress of an advanced UUO mouse, however, the KP-6T contains only 11 amino acids, and has the advantage of shorter peptide chain, easier synthesis, lower cost and easier absorption and distribution in vivo.

### BRIEF DESCRIPTION OF THE DRAWINGS

FIGS. 1A and 1B illustrate sirus red staining of a kidney of a UUO early administration model. FIG. 1A shows photographs being left: a sham operation group; middle: a UUO7d model group; and right: a UUO7d+KP-6T administration group. FIG. 1B is a graph showing a ratio of a collagenous fiber deposition area to a total area.

FIGS. 2A-2D illustrate immune staining and western blot of a kidney fibrosis index of mice in each group of the UUO early administration model. FIG. 2A is an immune staining diagram of a fibronectin, including Sham: a sham operation group, UUO7d: a model group, and UUO7d+KP-6T: an administration group; FIG. 2B is a western blot diagram of a fibronectin and an α-actin (α-SMA);

FIG. 2C is a relative quantitative statistical graph of an expression quantity of the fibronectin; and FIG. 2D is a relative quantitative statistical graph of an expression quantity of the α-actin.

FIGS. 3A-3D illustrate a level of an active-β-catenin protein and a mRNA level of a downstream target gene thereof in kidney tissues of medicament in each group of the UUO early administration model, wherein FIG. 3A is a western blot diagram of the active-β-catenin protein; FIG. 3B is a relative quantitative statistical graph of an expression quantity of the active-β-catenin protein; FIG. 3C illustrates a mRNA level of a downstream target gene I-type plasminogen activator inhibiting factor (PAI-1) of the active-β-catenin; and FIG. 3D illustrates a mRNA level of a downstream target gene Snail-1 of the active-β-catenin.

FIGS. 4A and 4B illustrate sirus red staining of a kidney of an advanced UUO administration model. In FIG. 4A, left: a sham operation group, middle: a UUO11d model group, and right: a UUO11d+KP-6T administration group. FIG. 4B shows a ratio of a collagenous fiber deposition area to a total area.

FIGS. 5A-5D illustrate immune staining and western blot of kidney fibrosis indexes of mice in each group of the advanced UUO administration model. FIG. 5A is an immune staining diagram of a fibronectin, including Sham: a sham operation group, UUO11d: a model group, and UUO11d+KP-6T: an administration group; FIG. 5B is a western blot diagram of a fibronectin and an α-actin (α-SMA); FIG. 5C is a relative quantitative statistical graph of an expression quantity of the fibronectin; and FIG. 5D is a relative quantitative statistical graph of an expression quantity of the α-actin.

FIGS. 6A-6D illustrate a level of an active-β-catenin protein and a mRNA level of a downstream target gene thereof in kidney tissues of mice in each group of the advanced UUO administration model, wherein FIG. 6A is a western blot diagram of the active-β-catenin protein; FIG. 6B is a relative quantitative statistical graph of an expression quantity of the active-β-catenin protein; FIG. 6C illustrates a mRNA level of a downstream target gene MMP7 of the active-β-catenin; and FIG. 6D illustrates a mRNA level of a downstream target gene PAI-1 of the active-β-catenin.

FIGS. 7A and 7B illustrate the effects of KP6T and KP6 in advanced UUO. FIG. 7A is a western blot diagram, wherein the fibronectin and the α-SMA are indexes related to the kidney fibrosis, Klotho reflects a severity of kidney tubular lesion, and the active-β-catenin is a key factor in activation of a Wnt signal pathway. FIG. 7B is a diagram illustrating sirus red staining and immumohistochemical staining of a fibronectin and an active-β-catenin of a kidney slice.

### DETAILED DESCRIPTION OF THE EMBODIMENTS

The present invention is further described in detail hereinafter with reference to the embodiments. It should also be understood that the following embodiments are only used to further illustrate the present invention and should not be construed as limiting the protection scope of the present invention. Some non-essential improvements and adjustments made by those skilled in the art according to the principles set forth in the present invention all fall within the protection scope of the present invention. The specific process parameters and the like of the following embodiments are only one example in a suitable range, that is, those skilled in the art can make choices in a suitable range through the description herein, and are not limited to the specific data of the following embodiments.

### Embodiment 1

An amino acid sequence of a KP-6T peptide fragment used in this embodiment is LQDAYGGWANR.

The KP-6T peptide fragment was synthesized by Nanjing Genscript Biotechnology Co., Ltd. The following experiments were performed.

1. Experimental Animals:

BALB/C mice, male and female, a weight ranging from 20 g to 22 g, and a SPF grade.

The animals were weighed and numbered. 18 healthy mice with a weight ranging from 20 g to 22 g were selected and randomly divided into 3 groups, with 6 mice in each group, including a sham operation group, a model group and an administration group.

2. Experimental Grouping

1) Sham operation group: at a room temperature, after mice were anaesthetized with 3% pentobarbital sodium at 1 ml/kg body weight, a left abdomen part of 2 cm to 3 cm was selected as an incision; and skin, a subcutaneous layer, a muscular layer and a peritoneum were cut layer by layer after local disinfection, and then were sutured layer by layer immediately after finding a left ureter. After local disinfection, the mice were verified and marked, and then placed in a corresponding mouse cage.

2) Model control group: anesthetization and disinfection were performed same as the sham operation group. Skin, a subcutaneous layer, a muscular layer and a peritoneum were cut layer by layer, and then were sutured layer by layer after finding a left ureter and performing a ligation at an upper ⅓ segment of the ureter. After local disinfection, the mice were verified and marked, and then placed in a corresponding mouse cage.

3) Administration group: anesthetization and disinfection were performed same as sham operation group. Skin, a subcutaneous layer, a muscular layer and a peritoneum were cut layer by layer, and then were sutured layer by layer after finding a left ureter and performing a ligation at an upper ⅓ segment of the ureter. After local disinfection, the mice were verified and marked, and then placed in a corresponding mouse cage.

3. Experimental Process

KP-6T or KP-6 water soluble powder was diluted with sterile 0.01 Mol glacial acetic acid solution to a storage concentration of 10 mg/ml. The mice of each experimental group were raised in different cages. The sham operation group was only observed. The model control group was only given 0.01 Mol glacial acetic acid solution by tail vein injection. The administration group was given glacial acetic acid solution containing KP-6T or KP-6 of 0.5 mg/kg of body weight by tail vein injection on the 1^{st} or 5^{th} day after UUO operation for 6 consecutive days. The mice in each group were euthanized after 7 days or 11 days of raising, left kidneys were taken, and then tissues were respectively fixed with 10% neutral formaldehyde and frozen with liquid nitrogen. The formaldehyde-fixed tissues were dehydrated, embedded, sliced and flaked, and then subjected to sirus scarlet staining and fibronectin immumohistochemical staining respectively. A protein was extracted after homogenization of the frozen tissues, and a fibrosis index, and expression levels of an active-β-catenin and a target gene thereof were detected by western blot.

4. Experimental Results

1) A degree of kidney tissue fibrosis was detected by sirus scarlet staining.

(I) KP-6T reduced collagen deposition in renal interstitium of UUO mice.

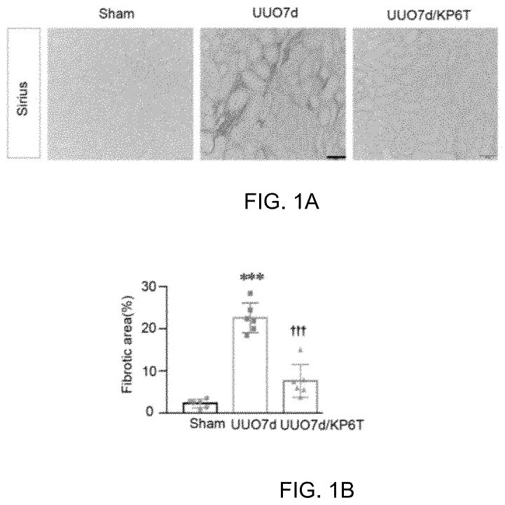

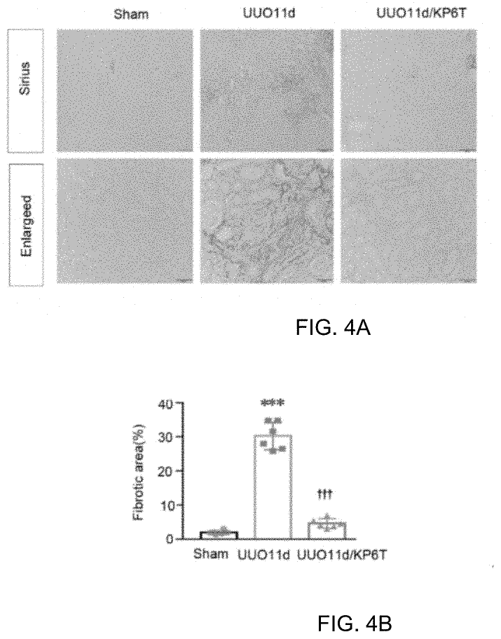

Experimental results were shown in FIG. 1 and FIG. 4, and collagen deposition in renal interstitium of mice in the administration group were significantly lower than that of mice in the model control group.

(II) KP-6T reduced kidney fibrosis of UUO mice.

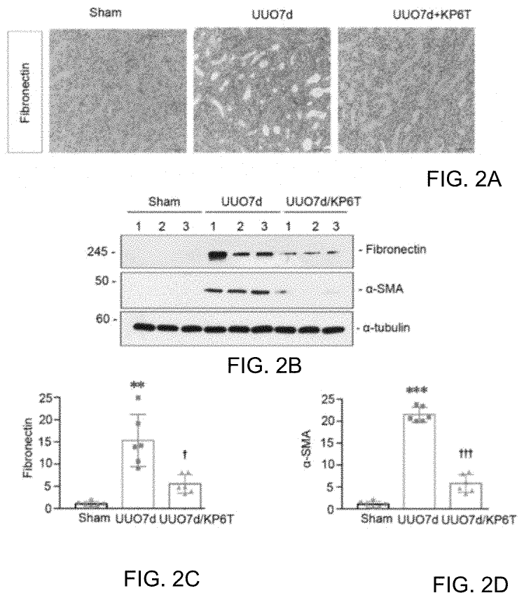

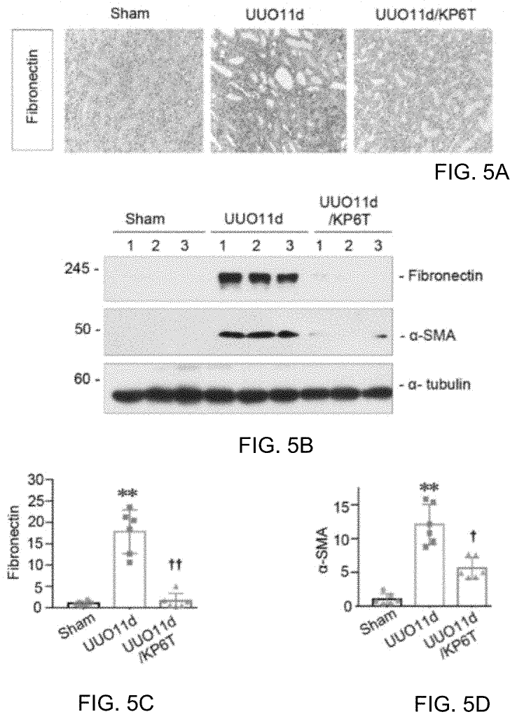

Experimental results were shown in FIG. 2 and FIG. 5. Compared with the model control group, levels of fibronectin and a smooth muscle actin α (α-SMA) in kidneys of mice in the administration group were significantly reduced.

(III) KP-6T inhibited an abnormally activated β-catenin signal pathway of UUO mice.

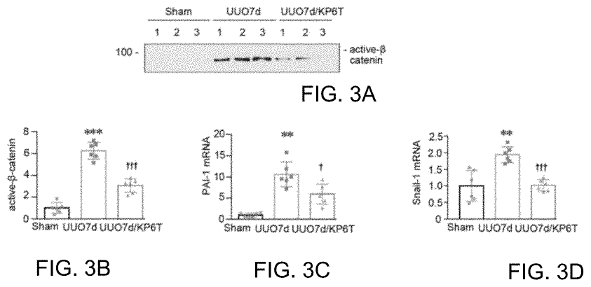

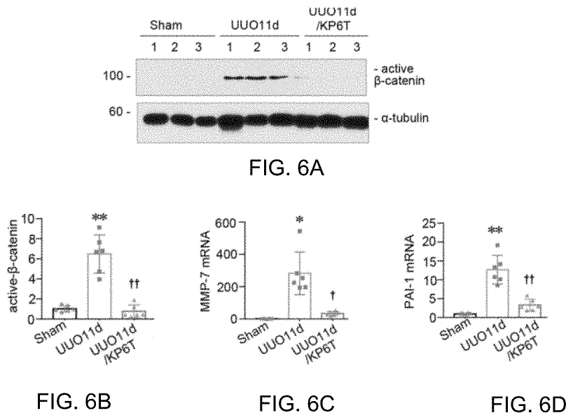

Experimental results were shown in FIG. 3 and FIG. 6. Compared with the model control group, expression levels of β-catenin protein and downstream target gene thereof in kidneys of mice in the administration group were significantly reduced.

(IV) Both KP6T and KP-6 could delay kidney fibrosis progress of an advanced UUO mouse.

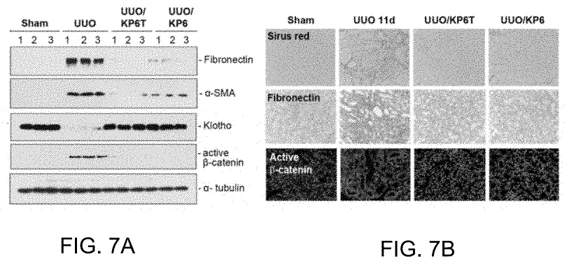

The experimental results were shown in FIG. 7. Compared with the model group, levels of a fibronectin and a smooth muscle actin α (α-SMA) in kidneys of mice given KP-6 and KP-6T were significantly reduced, deposition of extracellular matrix in renal interstitium were significantly reduced, protein Klotho expressed by healthy kidney tubular epithelial cells were recovered, and Wnt signal pathway were inhibited.

Above all, the KP-6T may significantly reduce the collagen deposition in the renal interstitium of the UUO mouse and the protein expression levels of the fibronectin and the α-SMA, and may significantly inhibit the abnormally activated β-catenin signal pathway in the CKD model. The medicament was administrated the 5^{th} day after UUO, which could also significantly inhibit the collagen deposition in the renal interstitium of the mouse and the expression levels of the fibronectin and the α-SMA protein. The KP-6T was indicated to be able to not only significantly delay the kidney fibrosis progress of the UUO mouse, but also block the renal interstitial fibrosis formed. Therefore, the KP-6T could be an effective new medicament for treating the CKD.

1. A micromolecule polypeptide, comprising any one of the following amino acid sequence:
a) LQDAYGGWANR (SEQ ID NO:1); and
b) an amino acid sequence obtained by modifying, substituting, deleting or adding at least one amino acid of the amino acid sequence shown in SEQ ID NO: 1.

2. A method for treating organ fibrosis, comprising administering a therapeutically effective amount of the micromolecule polypeptide according to claim 1 to a subject in need thereof, wherein the organ is any one or more selected from the group consisting of kidney, liver, lung and heart.

3. A method for treating chronic kidney disease, comprising administering a therapeutically effective amount of the micromolecule polypeptide according to claim 1 to a subject in need thereof.

4. A reagent for inhibiting expression levels of a β-catenin protein and a downstream target gene thereof, comprising an effective dose of the micromolecule polypeptide according to claim 1

5. The reagent according to claim 4, wherein the reagent further comprises a pharmaceutically acceptable ingredient.

6. A medicament for treating organ fibrosis, wherein the organ is any one or more selected from the group consisting of kidney, liver, lung and heart, and the medicament comprises an effective dose of the micromolecule polypeptide according to claim 1.

7. The medicament according to claim 6, wherein the medicament further comprises a pharmaceutically acceptable ingredient.

8. A medicament for treating chronic kidney disease, comprising an effective dose of the micromolecule polypeptide according to claim 1.

9. The medicament according to claim 8, wherein the medicament further comprises a pharmaceutically acceptable ingredient.


SEQUENCE LISTING (1 sequence)

SEQ ID NO: 1 - protein, 11 aa, Artificial Sequence
  Synthesized by Nanjing Genscript Biotechnology Co. Ltd.
LQDAYGGWANR